(19) United States
(12) Patent Application Publication
(10) Pub. No.: US 2008/0275118 A1
(43) Pub. Date: Nov. 6, 2008

(54) HEALTH AND COSMETIC COMPOSITION AND REGIME FOR STIMULATING HAIR GROWTH AND THICKENING ON THE HEAD, INCLUDING THE SCALP, EYELASHES, AND EYEBROWS, AND WHICH DISCOURAGES HAIR LOSS

(76) Inventors: Mari M. Shaw, Philadelphia, PA (US); Marc S. Cohen, Bala Cynwyd, PA (US); Nancy G. Swartz, Bala Cynwyd, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/137,799

(22) Filed: Jun. 12, 2008

(51) Int. Cl. A61K 31/201; A61P 27/06

(52) U.S. Cl. 514/559

(57) ABSTRACT

This invention relates to compositions and processes for stimulating the growth of mammalian hair comprising the topical application of compositions comprising a hair growth stimulating and/or hair loss prevention agent, and a hair and/or skin lightening and/or neutralization agent, in association with a topical pharmaceutical carrier. Unlike other products, the composition of the invention can be used with dyed/treated hair so as to not affect the color of the hair.

### BACKGROUND OF THE INVENTION

1. Field of Invention

This invention relates to compositions and processes for stimulating the growth of mammalian hair comprising the topical application of compositions comprising a hair growth stimulating and/or hair loss prevention agent, and a hair and/or skin lightening and/or neutralization agent, in association with a topical cosmetic, personal care product or pharmaceutical carrier, and methods for producing and using such compositions. Unlike other products, the composition of the invention will not affect the color of the hair, whether or not the hair has been dyed or treated, or the pigmentation of the skin which is touched by the compositions during the application process, or the iris of the eye, or the periorbital region.

2. Description of Related Art

There has been a long-felt need for methods of directly influencing hair growth, without affecting the color of the hair or the color of pigmentation of the skin to which the composition has been applied. This need arises for the treatment of alopecia and hair loss associated with, chemotherapy, or a disturbance of the hair renewal cycle during recovery from pregnancy, or during menopause, or during the process of recovery from stress, or inadequate nutrition.

Hair loss and baldness (alopecia) are common phenomena in mammals, including humans (see, for example, A. G. Messenger (1993) J. Investig. Dermatol. 101:4 S-9S; R. P. R. Dawber (1987) Dermatologica 175:23-28; D. G. Brodland, S. A. Muller (1991) Cutis 47:173-176; J. R. Spindler, J. L. Data (1992) Dermatol. Nurs. 4:93-99; A. K. C. Leung, W. L. M. Robson (1993) J. Roy. Soc. Health 113:252-256). Hair loss may be naturally occurring (primary alopecia) or it may be induced by chemical or physical agents (secondary alopecia). See, for example, M. B. Brodin (1987) Dermatol. Clin. 5:571-579; A Tosti, et al. (1994) Drug Saf. 10:310-317; H. J. Carson, et al. (1994) J. Cutan. Pathol. 21:67-70. Hair loss may also result from specific disease states, such as mange, or formation of scar tissue from bites, and with increasing age (D. A. Mehregan, et al. (1992) J. Am. Acad. Dermatol. 27:935-942; D. A. Slagle, T. A. Martin (1991) Am. Fam. Physician 43:2019-2024; L. V. Spencer, J. P. Callen (1987) Dermatol. Clin. 5:565-570. Hair loss is an extremely common condition in healthy adult male humans, and occurs frequently in adult female humans. In fact, some degree of alopecia on the vertex from puberty onwards is thought to be a universal phenomenon in both men and women (R. P. R. Dawber (1987) Dermatologica 175:23-28). Alopecia is also frequently observed in both pre- and post-pubertal patients as a side effect of anti-cancer chemotherapy (A. M. Hussein, et al. (1990) Science 249:1564-1566; B. W. Cline, (1984) Cancer Nursing 7:221-228; A. F. Hood (1986) Med. Clin. North Am. 70:187-209).

The physical phenomenon of hair loss may lead to psychological problems decreased social activity, and the development of psychological diseases. Patients, whose treatment threatens the likelihood of hair loss, such as cancer patients whose treatment requires chemotherapy, may lead to a refusal to accept treatment. As a result of the prevalence of hair loss, and its potentially devastating impact, there is immense interest in the development of effective clinical treatments, both to prevent hair loss and to stimulate regrowth of lost hair, without affecting the color of the hair, the color of the pigmentation of the skin to which the treatment is applied, the iris of the eye, and/or the periorbital tissue.

From this viewpoint, a large number of compositions comprising very diverse active principles, such as, for example, 2,4-diamino-6-piperidinopyrimidine 3-oxide or “minoxidil”, disclosed in U.S. Pat. Nos. 4,139,619 and 4,596,812, or its numerous derivatives, such as those disclosed, for example, in EP-0,353,123, EP-0,356,271, EP-0,408,442, EP-0,522,964, EP-0,420,707, EP-0,459,890 and EP-0,519,819, have already been proposed.

Clinical studies have demonstrated that PGF2α analogues have the property of bringing about the growth of body hairs and eyelashes in man and animals (Murray A. and Johnstone M. D., 1997, Am. J. Opht., 124(4), 544-547). In man, tests carried out on the scalp have shown that a prostaglandin E2 analogue (viprostol) has the property of increasing hair density (Roenigk H. H., 1988, Clinic Dermatol., 6(4), 119-121).

Furthermore, WO 98/33497 discloses pharmaceutical compositions comprising prostaglandins or prostaglandin derivatives intended to combat hair loss in mammals. Prostaglandins of the A2, F2α and E2 type are mentioned.

Many cosmetic and natural products sold over the counter and which rely mainly on coating the existing eyelashes are advertised as having the ability to grow eyelashes, see e.g., MAVALA® Eye-Lite Double Lash, Talika Eyelash Lipocils, Essential Eyelash Formula, Dream Lash®, and NTP Eyelash Enhancer. These products suffer from modest efficacy, can be inconvenient to administer, and may require permanent use to achieve and maintain the desired effect.

In the mid-to-late 90's, prostaglandin analogues, were introduced as intraocular pressure (IOP)-lowering drugs for use in patients with glaucoma and ocular hypertension; see, e.g., U.S. Pat. No. 6,262,105 which documents the use of a class of prostaglandins and their derivatives, for lowering intraocular pressure. One such analogue is commonly known as latanoprost, and latanoprost ophthalmic solution (marketed by Pharmacia & Upjohn under the trademark Xalatan®) is an FDA-approved drug. U.S. Pat. No. 5,889,052 (the '052 patent) documents the use of salts and esters of cloprostenol and fluprostenol and various analogues thereof, for the treatment of glaucoma and ocular hypertension. One such fluprostenol analogue is known as travoprost, and travoprost ophthalmic solution (marketed by Alcon Laboratories, Inc, under the trademark Travatan®) is also an FDA-approved drug. U.S. Pat. No. 5,688,819 identifies various prostaglandin analogues as potent ocular hypotensives and documents the use thereof as therapeutic agents. One such analogue is known as bimatoprost, and bimatoprost ophthalmic solution (marketed by Allergan, Inc. under the trademark Lumigan®) is also FDA-approved.

An important observation documented with the use of prostaglandin analogues in the treatment of glaucoma was the stimulating effects of the drugs on eyebrow and eyelash hair growth and pigmentation; more specifically, observations of increased eyelash growth, thickness, luster and pigmentation; see, e.g., U.S. Pat. No. 6,262,105. In view of this interesting finding, these and other Prostaglandin analogues have recently been further evaluated for their ability to promote hair growth, including eyelash growth/enhancement; see, e.g., Wolf, et al., 2003 Dermatology Online Journal 9(3): 7, and references cited therein.

While seemingly excellent candidates to become a drug of choice in promoting eyelash growth, there are several important and serious considerations to address when evaluating the long term safety and overall practicality of using prostaglandin analogues solely for the purpose of eyelash enhancement and/or hair growth. For example, additional observations associated with the use of prostaglandin analogues to treat glaucoma were darkening of the iris (colored part) and periorbital tissue of the treated eye (the change noticeable usually within several months or years from the start of treatment with the prostaglandin analog), as well as increased pigmentation of eyelashes and eyelid skin darkening. And, importantly, these changes to the iris, periorbital tissue, and eyelashes occur only in the eye being treated with the prostaglandin analog and may be permanent. As such, if only one eye is being treated, there exists the potential for increased brown pigmentation of the iris, periorbital tissue, and eyelashes in the treated eye only and thus, heterochromia between the eyes; as well as the potential for a disparity between the eyes in length, thickness, and/or number of eyelashes. However, the effect of the skin, hair, and/or eye color change is not immediately apparent, so that by the time the change of the color of the skin, hair, and/or eyes is noticed, it cannot be corrected.

Therefore, there exists in the art a need in the art for a composition comprising a hair growth stimulant, and/or hair growth stimulating agent and/or hair density increasing agent and/or hair loss prevention agent further comprising a skin and/or hair lightening agent, which will stimulate the growth and/or thickening of hair and/or density of hair, and also prevent darkening or change of the skin, hair, and/or eyes in response to the application of the hair growth stimulating agent and/or loss prevention agent.

The invention described herein provides for use of a hair growth stimulant, and/or hair growth stimulating agent and/or hair density increasing agent and/or hair loss prevention agent further comprising a skin and/or hair lightening agent, which will stimulate the growth and/or thickening of hair, and also prevent darkening or change of the skin, hair, and/or eyes in response to the application of the hair growth and/or thickening stimulating agent.

The invention described herein provide for use of hair growth stimulant, and/or hair growth stimulating agent and/or hair density increasing agent and/or hair loss prevention agents for eyelash, eyebrow, scalp, and other hair, enhancement and will be a tremendous benefit to those individuals having to deal with eyebrow, eyelash, scalp, or other hair loss, and/or thickening as a result of disease or medical treatment as well as the many individuals who seek eyebrow, eyelash, scalp, or other hair enhancement, for example, for cosmetic reasons.

In accordance with one embodiment of the invention, the method of using the hair growth stimulant, and/or hair growth stimulating agent and/or hair density increasing agent and/or hair loss prevention composition comprises topically treating the area of skin affected by hair loss. The method may comprise topical treatment as a single application, or it may comprise periodic treatment over an extended treatment time period as needed. Alternatively, the method may include a slow-release mechanism from a suitable carrier, or via any drug delivery mechanism. In accordance with an embodiment of the invention, the method may be applied to a subject who is receiving chemotherapy or radiotherapy and suffering hair loss. Such a situation may occur, for example, as a part of cancer treatment protocol, in order to induce hair follicle formation. The embodiment of the invention also includes the situation that the hair growth stimulator can be used for the subject who is going to receive chemotherapy or radiotherapy, to avoid hair loss, or who received chemotherapy or radiotherapy before and is suffering a permanent hair loss. Alternatively, the hair growth stimulator may be used on a subject who suffers hair loss from exposure to a toxic chemical or radioactive source. Such a situation may result from an industrial toxic chemical accident, explosion of chemical or nuclear plant, or accidental administration of toxic chemicals or toxic drugs. The composition and method of the invention may be applied to a subject who is receiving any type of therapy which may cause hair loss.

All References Cited Herein are Incorporated Herein by Reference in their Entireties.

### BRIEF SUMMARY OF THE INVENTION

The invention provides a topical composition for stimulating hair growth in a mammal comprising a hair growth stimulating agent and/or hair density increasing agent; a hair and/or skin lightening and/or neutralization agent; and a cosmetically, personal care, or pharmaceutically acceptable excipient, carrier or vehicle, wherein the composition has the effect of increasing length and/or thickness and/or density, and/or combination thereof, of the hair without darkening the hair and/or skin and/or iris of the mammal, and preventing hair loss.

The invention provides a topical composition for stimulating hair growth and/or slowing or preventing the loss of hair and/or increasing the thickness of the hair in a mammal comprising a hair growth stimulating agent and/or hair density increasing agent selected from the group consisting of prostaglandins, prostaglandin analogs, 15-PDGH inhibitors, and combinations thereof; a hair and/or skin lightening and/or neutralization agent; and a cosmetically or pharmaceutically acceptable excipient, carrier or vehicle, wherein the composition has the effect of increasing length and/or thickness and/or slowing or preventing loss, or any combination thereof, of the hair without darkening the hair and/or skin and/or iris of the mammal, and preventing hair loss.

In certain embodiments, the topical composition comprises a hair growth stimulating agent selected from the group consisting of prostaglandins, prostaglandin analogs, and combinations thereof.

In certain embodiments, the topical composition comprises a hair growth stimulating agent and/or hair density increasing agent selected from the group consisting of prostaglandin A2, prostaglandin F2, prostaglandin E1, prostaglandin E2, Arbaprostil, Carboprost, Enprostil, Bimatoprost, Bemeprost, Latanaoprost, Limaprost, Minoxidil, Misoprostol, Ornoprostil, Prostacyclin, Prostaglandin E1, Prostaglandin E2, Prostaglandin F2a, Rioprostil, Rosaprostol, Sulprostone, Travaprost, Trimoprostil, Viprostol, 15-PGDH inhibitors, and combinations thereof.

In certain embodiments, the topical composition comprises a lightening agent selected from the group consisting of hydroquinone, citric acid, perborate or persulfate salts, kojic acid, ascorbic acid, hydrogen peroxide, urea peroxide, melamine peroxide, sodium perborate, sodium percarbonate, sodium persulfate, and combinations thereof.

In certain embodiments, the hair is selected from the group consisting of head hair, scalp hair, eyelashes, eyebrows, mustaches, beards, chest, legs, arms, and combinations thereof.

The invention further provides a composition in a form selected from the group consisting of application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like, bath and shower gels, shampoos, conditioners, cream rinses, hair dyes and coloring products, leave-on conditioners, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form, gels, patches, creams, sprays, ointments, lotions, emulsions, colloids, solutions, suspensions, powders and the like, and combinations thereof.

In certain embodiments, the topical composition comprises at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV blocking agents, cosmetic and pharmaceutical active principles other than the compounds of formula (I), and combinations thereof.

In certain embodiments, the topical composition further comprises at least one other active principle selected from the group consisting of proteins, protein hydrolysates, amino acids, polyols, urea, allantoin, sugars and sugar derivatives, plant extracts, hydroxy acids, retinol or tocopherol derivatives, 5-(n-octanoyl)salicylic acid, esters of hydroxy acids, phospholipids, and combinations thereof.

In certain embodiments the topical composition comprises at least one additional ingredient which is a cleaning agent, hair conditioning agent, skin conditioning agent, hair styling agent, antidandruff agent, hair growth promoter, perfume, sunscreen, sunblock, pigment, moisturizer, film former, hair color, make-up agent, detergent, pharmaceutical, thickening agent, emulsifier, humectant, emollient, antiseptic agent, deodorant active, dermatologically acceptable carrier, surfactant, abrasive, absorbent, fragrance, coloring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, addition peptide, buffering agent, bulking agent, chelating agent, cosmetic biocide, denaturant drug astringent, external analgesic, polymer, quat, substantivity increasing agent, opacifying agent, pH adjuster, propellant, reducing agent, sequestrant, skin bleaching agent, skin lightening agent, skin-conditioning agent, skin soothing agent, skin healing agent, aloe vera, pantothenic acid and derivative thereof, allantoin, bisabolol, dipotassium glycyrrhizinate, skin treating agent, thickener, or a vitamin or derivative thereof and mixtures or combinations thereof.

In certain embodiments the topical composition further comprises a plurality of additional ingredients and wherein at least one of said additional ingredients is a dermatologically acceptable carrier.

The invention further provides a delivery device for the targeted application to the hair of a mammal a therapeutically effective amount of the composition.

The invention further provides the delivery device, wherein the device is for the application of the composition to eyelashes, eyebrows, and/or scalp.

The invention further provides a method for stimulating hair growth in a mammal comprising the application to mammalian skin an effective amount of the composition.

The invention further provides a method for stimulating hair growth in a mammal comprising the application to mammalian skin of an effective amount of a composition comprising a hair growth stimulating agent and/or hair density increasing agent; a hair and/or skin lightening and/or neutralization agent; and a cosmetically, personal care, or pharmaceutically acceptable excipient, carrier or vehicle, wherein the composition has the effect of increasing length and/or thickness and/or both density, and/or combination thereof, of the hair without darkening the hair and/or skin and/or iris of the mammal, and preventing hair loss.

In certain embodiments, the method for increasing the thickness and length of hair comprises applying the composition to the eyelids in the immediate vicinity of the normal lash line, or on the eyelashes.

In certain embodiments, the method for stimulating the growth of natural eyelashes in a human subject comprises: applying to the base of the eyelash, or on the eyelashes, a therapeutically effective amount of the composition.

In certain embodiments, the method for stimulating the growth of eyelashes in a patient comprises: applying to the base of the eyelash, or on the eyelashes, a therapeutically effective amount of the composition, wherein the patient is being treated for cancer.

In certain embodiments, the method for stimulating the growth of eyelashes in a patient comprises: applying to the base of the eyelash, or on the eyelashes, a therapeutically effective amount of the composition, wherein the patient suffers from alopecia.

In certain embodiments, the method for increasing the thickness, density and/or length of hair of an eyebrow or eyebrows comprises applying to the eyebrow or eyebrows the composition.

In certain embodiments, the method for stimulating the growth of a natural eyebrow or eyebrows in a human subject comprises: applying to the eyebrow or eyebrows a therapeutically effective amount of the composition.

In certain embodiments, the method for stimulating the growth of an eyebrow or eyebrows in a patient comprises: applying to the eyebrow or eyebrows a therapeutically effective amount of the composition, wherein the patient is being treated for cancer.

In certain embodiments, the method for stimulating the growth of an eyebrow or eyebrows in a patient comprises: applying to the eyebrow or eyebrows a therapeutically effective amount of the composition, wherein the patient suffers from alopecia.

### DETAILED DESCRIPTION OF THE INVENTION

### Cosmetics

“Cosmetics,” as used herein, include without limitation, application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like.

### Personal Care Products

“Personal care products” include, without limitation, bath and shower gels, shampoos, conditioners, cream rinses, hair dyes and coloring products, leave-on conditioners, sun tanning lotions and creams and sprays, sunscreens and sunblocks, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form.

### Pharmaceutical Preparations

Pharmaceutical preparations in accordance with the present invention include, without limitation, carriers for dermatological purposes, including topical and transdermal application of pharmaceutically active ingredients. These can be in the form of gels, patches, creams, sprays, ointments, lotions, emulsions, colloids, solutions, suspensions, powders and the like.

### Hair

As used herein the term “hair” is meant to encompass all mammalian hair. While in humans the hair whose growth is to be regulated is typically located upon the head, scalp, eyebrows, eyelids, eyelashes, mustaches, beards, chest, arms, legs, and the pubic area, the inventive method and compositions described herein may be applied to hair located anywhere on the body.

As used herein, the terms “hair growth,” “regulating hair growth,” “hair growth regulation,” and “regulating the growth of hair,” are meant to include: stimulating hair growth; stimulating hair thickening; preventing, reducing, arresting and/or retarding the loss of hair; preventing, reducing, arresting and/or retarding the thinning of hair; increasing the rate of hair growth; inducing the formation of a greater number of hair strands; increasing the diameter of the hair strand; lengthening the hair strand; changing the hair follicle from vellus follicle to terminal follicle; inducing the formation of vellus follicles.

### Hair Growth Stimulating Agents

The methods and compositions of the instant invention may comprise any agent which stimulates hair growth and/or prevents hair loss, or thinning, including but are not limited to polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, small molecules, siNA, siRNA, dsRNA, dsDNA, anti-senseDNA, nucleic acids, antibodies, polyclonal antibodies, monoclonal antibodies, structural analogs, synthetic molecules, other natural molecules, small molecules, and combinations thereof, which may be employed to achieve the therapeutic effect on stimulation of hair growth and/or prevention of loss of hair in the types of tissues discussed above, e.g., the eyelid, eyebrow, scalp, and skin.

Non-limiting examples of hair growth stimulant, and/or hair growth stimulating agent and/or hair density increasing agent and/or hair loss prevention agents include prostaglandins or prostaglandin analogues which may be employed to achieve the therapeutic effect on stimulation of hair growth in the types of tissues discussed above, e.g., the eyelid, eyebrow, scalp, and skin. Non-limiting examples of prostaglandins are those of the A, F and E types. To minimize side effects, such as irritation and redness of the skin, it may be advantageous to use prostaglandin derivatives or analogues which have been found to exert fewer side effects, such as phenyl- and other ring-substituted prostaglandin derivatives. On the other hand, increased hyperemia experienced with other substituents may be more beneficial in causing increased vasodilation, resulting in hair growth. Prostaglandin derivatives that exhibit high pharmacological activity and no or only very small side effects, such as 13,14-dihydro-15-dehydro-17-phenyl-18,19,20-trinor-PGF2α and its carboxylic acid esters. Further non-limiting examples of suitable prostaglandins agonists or antagonists include latanoprost and those described in WO 98/33497, Johnstone, published Aug. 6, 1998, WO 95/11003, Stjernschantz, published Apr. 27, 1995, JP 97-100091, and Ueno, JP 96-134242, Nakamura.

Examples of pharmaceutical hair growth stimulating agent and/or hair growth stimulating agent and/or hair density increasing agent and/or hair loss prevention agents include, but are not limited to prostaglandin A2, prostaglandin F2α, prostacyclin, prostaglandin E1, prostaglandin E2,7-thiaprostaglandin E1,16,17,18,19,20-pentanol-15-cyclohexyl-7-thiaprostaglandin E1, 16,17,18,19,20-pentanol-15-cyclopentyl-7-thiaprostaglandin E1,16,16-dimethyl-7-thiaprostaglandin E1,17,20-dimethyl-7-thiaprostaglandin E1,16,17,18,19,20-pentanol-15-cyclohexyl-δ2-7-thiaprostaglandin E1,16,16-dimethyl-δ2-prostaglandin E1, 7-fluoroprostacyclin, 5-fluoroprostacyclin, 16,17,18,19,20-pentanol-15-cycrohexylprostacyclin or 16,17,18,19,20-pentanol-15-cycropentylprostacyclin. Other examples of prostaglandins and prostaglandin analogues which may be used in the instant invention include, but are not limited to, Arbaprostil, Carboprost, Enprostil, Bimatoprost, Bemeprost, Latanaoprost, Limaprost, Misoprostol, Minoxidil, Omoprostil, Prostacyclin, Prostaglandin E1, Prostaglandin E2, Prostaglandin F2α, Rioprostil, Rosaprostol, Sulprostone, Travaprost, Trimoprostil, and Viprostol. Other examples of hair growth stimulating and/or hair loss prevention agents include, but are not limited to 15-hydroxyprostaglandin dehydrogenase (15-PGDH) inhibitors, including but not limited to pyrazolecarboxamide compounds, tetrazole compounds, and 2-alkylideneaminooxyacetamide compounds (see U.S. Patent Application Publication Nos. 20060026775, 2004/0052760, and 2004/0235831). Prostaglandins and derivatives thereof are described in WO 98/33497, WO 95/11003, JP 97-100 091 and JP 96-134 242, prostaglandin receptor agonists or antagonists, the non-prostanoic prostaglandin analogues as described in EP 1 175 891, EP 1 175 890, WO 01/74307, WO 1/74313, WO 01/74314, WO 01/74315 or WO 01/72268, and mixtures thereof.

Lightening and/or Neutralization Agents

The methods and compositions of the instant invention may comprise any agent which prevent darkening or change of the skin, hair, and/or eyes in response to the application of the hair growth stimulating agent and/or hair loss prevention agent. The methods and compositions of the instant invention may comprise any agent which will neutralize any darkening or change of the skin, hair, and/or eyes in response to the application of the hair growth stimulating agent and/or hair loss prevention agent. Lightening or bleaching of hair and/or skin is discussed in the publication Perfumes, Cosmetics and Soaps, Vol. III Modern Cosmetics, Chapter 7, by W. A. Poucher, revised by George M. Howard, Eighth Edition (1976) (J. Wiley and Sons, New York); and the publication Harry's Cosmeticology, Vol I by R. G. Harry, Sixth Edition (1973) (Leonard Hill Books, London).

Examples of hair and/or skin lightening and/or neutralization agents or bleaching agents include but are not limited to hydroquinone, hydroquinone monobenzyl ether, and hydroquinone monoethyl ether, which act as substitute substrate for the melanization enzyme tyrosinase and may be included in certain peels to enhance the skin lightening ability of the peel. The addition of citric acid and malic acid in peels containing hydroquinone or its derivatives serve to act as antioxidants to help stabilize the hydroquinone in the peel and to enhance the penetration and the efficacy of the hair and/or skin lightening and/or neutralization agent.

Examples of hair and/or skin lightening and/or neutralization agents or bleaching agents also include but are not limited to perborate or persulfate salts, citric acid, hydroquinone, kojic acid ascorbic acid, and combinations thereof.

Where the composition is to be in contact with human keratinous tissue, the composition may include additional components which are suitable for application to keratinous tissue, that is, when incorporated into the composition are suitable for use in contact with human keratinous tissue without undue toxicity, incompatibility, instability, allergic response, and the like within the scope of sound medical judgment. The CTFA Cosmetic Ingredient Handbook, Second Edition (1992) describes a wide variety of non-limiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof, and combinations thereof.

While the exact composition of the hair and/or skin lightening and/or neutralization agents or bleaching agents will vary depending upon the specific end use, an essential component of the system is the oxidizing or bleaching agent. Useful oxidizing agents for compositions of this invention include but are not limited to hydrogen peroxide, urea peroxide, melamine peroxide, sodium perborate, sodium percarbonate and sodium persulfate.

The quantity of oxidizing agent used will vary according to the particular agent employed and the specific end use of the composition. Generally, the oxidizing agent will be present in the range from about 0.5% to about 20% by weight based upon the total weight of the lightening or bleaching system. When hydrogen peroxide is employed as the oxidizing agent, a non-limiting example of the range is from about 2% to about 4% by weight, based upon the total weight of the composition.

The hair and/or skin lightening and/or neutralization agents or bleaching agents of the subject composition is an aqueous based composition with the terminology being used in its usual generic sense as inclusive of any water-containing compositions embodied in the invention. Thus, this includes true solutions or mixtures of materials dissolved or dispersed in the aqueous medium. The amount of water present in this component can vary over a wide range depending in a large measure on the quantity of the other ingredients.

Various organic solvents may also be present in the composition for the purpose of solubilizing any of the components which may be insufficiently soluble in water. Generally, the solvent selected is such as to be miscible with water and innocuous to the skin. Suitable solvents include, for example, ethanol, isopropanol, glycerine, ethylene glycol, propylene glycol, ethylene glycol monoethyl ether, diethylene glycol, diethylene glycol monoethyl ether, etc.

Various thickening agents may also be incorporated in the composition component of the subject composition. Useful thickening agents including sodium alginate, gum arabic, cellulose derivatives, such as methyl cellulose, hydroxyethyl cellulose, etc. Likewise, inorganic thickening agents such as bentonite, talc and kaolin may be useful.

The composition may have an antioxidant incorporated in it, and useful antioxidants for this purpose include sodium sulphite, thioglycollic acid, sodium hydrosulfite, and ascorbic acid. Sequestering agents may also be present in the bleaching system. Useful sequestering agents include ethylenediamine tetraacetic acid and its various sodium salts, tetrasodium pyrophosphate and the like. Surfactants, additionally, may be present in the bleaching system and useful surfactants include the anionic, non-ionic and cationic type. Also, the bleaching system may contain dyes, perfumes, and conditioners as are conventionally employed in the art.

### Topical Formulations

The term “topical” as employed herein relates to the use of a compound, derivative or analogue as described herein, incorporated in a suitable pharmaceutical carrier, and applied at the site of thinning hair for exertion of local action. Accordingly, such topical compositions including those forms in which the compound is applied externally by direct contact with the skin surface to be treated. Conventional forms for this purpose include ointments, liniments, creams, shampoos, lotions, pastes, jellies, sprays, aerosols, soaps, and the like, and may be applied in patches or impregnated dressings depending on the part of the body to be treated. The term “ointment” embraces formulations (including creams) having oleaginous, absorption, water-soluble and emulsion-type bases, e.g., petrolatum, lanolin, polyethylene glycols, as well as mixtures of these.

For topical use on the skin, eyelids, eyebrows, the active hair growth stimulating agents as well as their derivatives and analogues, including esters and salts, can be formulated in aqueous solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and the hair growth stimulating agents to be used, various amounts of the drug and different dose regimens may be employed. Typically, the daily amount of the hair growth stimulating and/or hair loss prevention agent may be about 0.1 ng to about 100 mg, or about 1 ng to about 100 μg, but may be out of that range.

For topical use, the hair growth stimulating and/or hair loss prevention agent can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied is in the range of about 0.1 ng to about 100 mg per day, or about 1 ng to about 10 mg per day, or about 10 ng to about 1 mg per day depending on the hair growth stimulating agent and the formulation. To achieve the daily amount of medication depending on the formulation, the hair growth stimulating and/or hair loss prevention agent may be administered once or several times daily, with or without antioxidants.

The composition or regime for application can be provided in the form of a hair care lotion, at various times, including but not limited to for example for daily or twice-weekly application, of a shampoo or of a hair conditioner, in particular for twice-weekly or weekly application, of a liquid or solid soap for cleaning, for daily application, of a product for shaping the hairstyle (lacquer, hairsetting product, styling gel), of a treatment mask, of a cream or of a foaming gel for cleaning the hair. It can also be provided in the form of a hair dye or mascara to be applied with a brush or comb.

Furthermore, for application to the eyelashes or body hairs, the composition and regime to which the invention relates can be provided in the form of a pigmented or nonpigmented mascara, applied with a brush pencil or other applicator device to the eyelashes or alternatively to the beard or moustache hairs, or scalp.

According to a specific embodiment, the composition and regime according to the invention is provided in the form of a hair cream or lotion, shampoo, hair conditioner, hair mascara, or other hair coloring device, or of a mascara eyeliner, or pencil.

Non-limiting examples of topical products can include, without limitation, application stick, mascara, eyebrow coloring products, eye shadow or other eye lid coloring products, eyeliner, make-up removal products, antiaging products, facial or body powder, nail polish, mousse, sprays, styling gels, nail conditioner, bath and shower gels, shampoos, conditioners, cream rinses, hair dyes and coloring products, hair conditioners, sun tanning lotions and creams and sprays, sunscreens and sunblocks, skin conditioners, cold creams, moisturizers, hair sprays, soaps, body scrubs, exfoliants, astringents, depilatories and permanent waving solutions, antidandruff formulations, antisweat and antiperspirant compositions, shaving, preshaving and after shaving products, moisturizers, deodorants, cold creams, cleansers, skin gels, and rinses.

Furthermore, the topical product can be applied topically through the use of a patch or other delivery device. Delivery devices can include, but are not limited to, those that can be heated or cooled, as well as those that utilize iontophoresis or ultrasound.

For instance, the topical product can be applied, for example, by applying a composition in the form of a skin lotion, clear lotion, milky lotion, cream, gel, foam, ointment, paste, emulsion, spray, conditioner, tonic, cosmetic, application stick, pencil, foundation, nail polish, after-shave, or the like which is intended to be left on the skin or other keratinous tissue (i.e., a “leave-on” composition). After applying the composition to the keratinous tissue (e.g., skin), it in one embodiment, it is left on for a period of at least about 15 minutes, or at least about 30 minutes, or at least about 1 hour, or for at least several hours, e.g., up to about 12 hours. In one embodiment, the topical product is left on overnight. In another embodiment, the topical product is left on all day. Any part of the external portion of the face, hair, and/or nails can be treated, (e.g., face, lips, under-eye area, eyelids, scalp, neck, torso, arms, legs, chest, hands, legs, feet, fingernails, toenails, scalp hair, eyelashes, eyebrows, etc.)

Any suitable method can be used to apply the topical product, including but not limited to for example using the palms of the hands and/or fingers or a device or implement (e.g., a cotton ball, swab, pad, applicator pen, spray applicator, eyebrow brush, eyebrow brush pencil, pencil, mascara brush, etc.) Another approach to ensure a continuous exposure of the keratinous tissue to at least a minimum level of the topical product is to apply the compound by use of a patch applied, e.g., to the face. The patch can be occlusive, semi-occlusive or non-occlusive, and can be adhesive or non-adhesive. The topical product can be contained within the patch or be applied to the skin prior to application of the patch. The patch can also include additional actives such as chemical initiators for exothermic reactions such as those described in PCT application WO 9701313, and in U.S. Pat. Nos. 5,821,250, 5,981,547, and 5,972,957 to Wu, et al. The patch can be left on the for any suitable period of time. For example, a period of at least about 5 minutes, or at least about 15 minutes, or at least about 30 minutes, or at least about 1 hour, or at night as a form of night therapy, or in another embodiment all day.

The topical product can comprise any suitable desired materials. For instance, such materials can be selected from the group consisting of sugar amines (e.g., N-acetylglucosamine), vitamin B3 compounds, sodium dehydroacetate, dehydroacetic acid and its salts, phytosterols, soy derivaties (e.g., equol and other isoflavones), niacinamide, phytantriol, farnesol, bisabolol, salicylic acid compounds, hexamidines, dialkanoyl hydroxyproline compounds, flavonoids, N-acyl amino acid compounds, retinoids (e.g., retinyl propionate), water-soluble vitamins, ascorbates (e.g., vitamin C, ascorbic acid, ascorbyl glucoside, ascorbyl palmitate, magnesium ascorbyl phosphate, sodium ascorbyl phosphate), particulate materials, sunscreen actives, anti-cellulite agents, butylated hydroxytoluene, butylated hydroxyanisole, their derivatives, and combinations thereof. Other examples include cationic polymers, conditioning agents (hydrocarbon oils, fatty esters, silicones), anti-dandruff agents, suspending agents, viscosity modifiers, dyes, nonvolatile solvents or diluents (water soluble and insoluble), pearlescent aids, foam boosters, surfactants, nonionic cosurfactants, pediculocides, pH adjusting agents, perfumes, preservatives, chelants, chelating agents, proteins, UV absorbers, pigments, other amino acids, and other vitamins.

For instance, topical products for use herein may comprise one or more vitamins and/or amino acids such as: water soluble vitamins such as vitamin B1, B2, B6, B12, C, pantothenic acid, pantothenyl ethyl ether, panthenol, biotin, and their derivatives, water soluble amino acids such as asparagine, alanine, indole, glutamic acid and their salts, water insoluble vitamins such as vitamin A, D, E, and their derivatives, water insoluble amino acids such as tyrosine, tryptamine, and their salts.

Topical products may also contain one or more pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. Names. The topical products may also contain antimicrobial agents which are useful as cosmetic biocides and antidandruff agents including: water soluble components such as piroctone olamine, water insoluble components such as 3,4,4′-trichlorocarbanilide (trichlosan), triclocarban and zinc pyrithione.

### Additional Ingredients

In addition to the hair growth stimulating and/or hair loss prevention agent, and hair and/or skin lightening and/or neutralization agent, the compositions of the invention may include various other and additional ingredients, which may be active, functional, conventionally used in cosmetic, personal care or topical/transdermal pharmaceutical products or otherwise. Of course, a decision to include an additional ingredient and the choice of specific additional ingredients depends on the specific application and product formulation. Also, the line of demarcation between an “active” ingredient and an “inactive ingredient” is artificial and dependent on the specific application and product type. A substance that is an “active” ingredient in one application or product may be a “functional” ingredient in another, and vice versa. A particular ingredient might provide substantivity in one formulation, facilitate transdermal application in another, and merely provide proper viscosity in a third. Which of these is functional and which is active is subject to debate. But, regardless of the outcome, the material in question would qualify as an additional ingredient in accordance with the present invention.

Thus, the compositions of the invention may include one or more additional ingredients, which provide some benefit to the object of the composition. Such additional ingredients may include one or more substances such as, without limitations, cleaning agents, hair conditioning agents, skin conditioning agents, hair styling agents, antidandruff agents, hair growth promoters, perfumes, sunscreen and/or sunblock compounds, pigments, moisturizers, film formers, hair colors, make-up agents, detergents, pharmaceuticals, thickening agents, emulsifiers, humectants, emollients, antiseptic agents, deodorant actives, dermatologically acceptable carriers, surfactants, hair straightening agents, hair coloring agents, hair bleaching agents, and combinations thereof.

The compositions of the present invention generally contain at least one additional ingredient. The compositions of the present invention may contain a plurality of additional ingredients as well.

Where the composition is to be in contact with human keratinous tissue, the additional ingredients should be suitable for application to keratinous tissue, that is, when incorporated into the composition they are suitable for use in contact with human keratinous tissue (hair, nails, skin, lips) without undue toxicity, incompatibility, instability, allergic response, and the like within the scope of sound medical judgment. The CTFA Cosmetic Ingredient Handbook, Ninth Edition (2002) describes a wide variety of nonlimiting cosmetic and pharmaceutical ingredients commonly used in the skin care industry, which are suitable for use as additional ingredients in the compositions of the present invention. Non-limiting examples of these additional ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, vitamins and derivatives thereof, hair straightening agents, hair coloring agents, hair bleaching agents, and combinations thereof.

In any embodiment of the present invention, however, the additional ingredients useful herein can be categorized by the benefit they provide or by their postulated mode of action. However, it is to be understood that the additional ingredients useful herein can in some instances provide more than one benefit or operate via more than one mode of action. Therefore, classifications herein are made for the sake of convenience and are not intended to limit the additional ingredients to that particular application or applications listed.

### Cosmetic or Personal Care Products

The compositions of the invention may be used in various cosmetic and/or personal care products, for example, skin care, hair care, nail care, facial and body care and sunscreen compositions, such as lotions, gels, sprays, and the like, hand cleaners, bath compositions, suntan oils, anti-perspirant compositions, perfumes and colognes, cold creams, hair sunscreen compositions, pre-shaves, deodorants, topical pharmaceutical ointments, skin moisturizers, facial cleansers, cleansing creams, skin gels, shampoos, hair conditioners, detergents, household cleaning products, make-up products, lipstick products, mascara, hair coloring products, hair straightening agents, hair coloring agents, hair bleaching agents, and combinations thereof. Therefore, in addition to any of the above cited skin care or hair care peptides and other actives, the cosmetic compositions described in the present invention may often include as an additional ingredient a dermatologically acceptable carrier. The form of the composition and the final product may be any of the following: liquids, gels, creams, water-in-oil and oil-in-water, and silicone emulsions, foams, and solids; they may be clear or opaque; and may be formulated as both aqueous and non-aqueous preparations, including but not limited to topical preparations.

To realize the invention in any of these physical forms, further substances, agents and compounds are useful although not always necessary such as Conditioning Agents, Structuring Agents and Thickening Agents. These compounds sometimes also have the role of adjuvant and sometimes the role of additional ingredient.

The nature of the dermatologically acceptable carrier, the nature of the final product, and the methods of preparing those need not be described here in detail; many examples can be found in the available literatures, such as PCT application No. WO 00/62743 filed by Larry R. Robinson et al. on Apr. 19, 2000, published on Oct. 26, 2000, or, more generally, in Milady's Standard Textbook of Cosmetology 2000, (Delmar Learning) or in Formulation Technology: Emulsions, Suspensions, Solid Forms by Hans Mollet, Arnold Grubenmann and Helen Payne, published by John Wiley & Sons (Jan. 23, 2001), or in Chemistry and Technology of the Cosmetics and Toiletries Industry by Clifford Williams Schmitt, Kluwer Academic Publishers, Dordrecht July 1996, all hereby incorporated. Fiedler's Encyclopedia of Excipients, fifth edition, Edition Cantor Verlag Aulendorf, 2002 is also a useful guide for the formulator skilled in the art of developing cosmetic carriers. All ingredients listed therein may in one way or another be combined to form a dermatologically acceptable carrier and/or used as an additional ingredient for the cosmetic compositions of the invention.

In most instances, the additional ingredients will include a dermatologically acceptable carrier either alone or in combination with still other additional ingredients. The amounts of additional ingredients may range from about 99.5% to about 99.99999%, or from about 99.9% to about 99.9999%, or from about 99.99% to about 99.999%, of the composition, but may be outside these ranges. In short, it is the balance of the composition. If carriers (either singularly, such as water, or complex cosolvents) are used, they may make up the entire balance of the compositions.

The carrier can be in a wide variety of forms. For example, emulsion carriers, including, but not limited to, oil-in-water, water-in-oil, water-in-oil-in-water, and oil-in-water-in-silicone emulsions, are useful herein.

Non-limiting examples of carriers include carriers which contain an emulsion such as oil-in-water emulsions, water-in-oil emulsions, and water-in-silicone emulsions. As will be understood by the skilled artisan, a given component will distribute primarily into either the water or oil/silicone phase, depending on the water solubility/dispersibility of the component in the composition.

Emulsions according to the present invention generally contain a solution as described above and a lipid or oil. Lipids and oils may be derived from animals, plants, or petroleum and may be natural or synthetic (i.e., man-made). Emulsions may also contain a humectant, such as glycerin. Emulsions may further contain from about 0.01% to about 10%, or from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986).

The emulsion may also contain an anti-foaming agent to minimize foaming upon application to the keratinous tissue. Anti-foaming agents include but are not limited to high molecular weight silicones and other materials well known in the art for such use.

Lotions and creams according to the present invention generally contain a solution carrier system and one or more emollients. Lotions and creams may for example contain from about 1% to about 50%, or from about 1% to about 20%, of emollient; from about 50% to about 90%, or from about 60% to about 80% of water; and the tetrapeptide and tripeptide mixture and the optional additional skin care active (or actives) in the above described amounts. Creams are generally thicker than lotions due to higher levels of emollients or higher levels of thickeners.

Ointments of the present invention may contain a simple carrier base of animal or vegetable oils or semi-solid hydrocarbons (oleaginous); absorption ointment bases which absorb water to form emulsions; or water soluble carriers, e.g., a water soluble solution carrier. Ointments may further contain a thickening agent, such as described in Sagarin, Cosmetics, Science and Technology, 2nd Edition, Vol. 1, pp. 72-73 (1972), incorporated herein by reference, and/or an emollient. For example, an ointment may contain from about 2% to about 10% of an emollient; from about 0.1% to about 2% of a thickening agent; and the tetrapeptide and tripeptide mixture and the optional additional skin care active (or actives) in the above described amounts.

### Administration

Typically, the composition may be applied repeatedly for a sustained period of time topically on the part of the body to be treated, for example, the eyelids, eyebrows, skin or scalp. The dosage regimen will generally involve regular, such as daily, administration for a period of treatment of at least one month, or at least three months, or at least six months.

Alternatively, the composition may be applied intermittently, or in a pulsed manner. Accordingly, an alternative embodiment of the invention is to apply the composition on an intermittent or pulsed dosage schedule. For example, the composition of the invention may be used for two or more days, stopped, then restarted again at a time from between 2 weeks to 3 months later, and at even more long-spaced intervals in the case of the scalp.

For topical use on the eyelids or eyebrows, the composition can be formulated in aqueous solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and hair growth stimulant, and/or hair growth stimulating agent and/or hair density increasing agent and/or hair loss prevention agent to be used, various amounts of the drug and different dose regimens may be employed. Typically, the daily amount of hair growth stimulant, and/or hair growth stimulating agent and/or hair density increasing agent and/or hair loss prevention agent for treatment may be about 0.1 ng to about 100 mg, or about 1 ng to about 100 μg.

For topical use on the skin and the scalp, the composition can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied is in the range of about 0.1 ng to about 100 mg per day, or about 1 ng to about 10 mg per day, or about 10 ng to about 1 mg per day depending on the hair growth stimulant, and/or hair growth stimulating agent and/or hair density increasing agent and/or hair loss prevention agent and the formulation. To achieve the daily amount of medication depending on the formulation, the hair growth stimulant, and/or hair growth stimulating agent and/or hair density increasing agent and/or hair loss prevention agent may be administered once or several times daily with or without antioxidants.

The invention will be illustrated in more detail with reference to the following Examples, but it should be understood that the present invention is not deemed to be limited thereto.

### EXAMPLES

### Example 1

Cosmetic Composition

| Ingredient | Amount, weight percent (wt %) |
|---|---|
| Latanoprost | 0.005 |
| Hydroquinone | 0.5 |
| Monobasic sodium phosphate | 0.05 |
| Dibasic sodium phosphate (anhydrous) | 0.15 |
| Benzalkonium chloride | 0.02 |
| Sodium chloride | 0.75 |
| HCl and/or NaOH | pH 6.7 |
| Disodium EDTA | 0.05 |
| Purified water | q.s. to 100% |

### Example 2

Cosmetic Composition

| Ingredient | Amount, weight percent (wt %) |
|---|---|
| Latanoprost | 0.005 |
| Hydroquinone | 0.5 |
| Titanium dioxide | 2.0 |
| Sodium polyphosphate | 0.05 |
| Purified water | q.s to 100% |
| Polyoxyethylene sorbitan monooleate | 0.5 |
| Glycerin | 3.0 |
| Sodium carboxymethyl cellulose | 1.0 |
| Bentonite | 0.5 |
| Polymer emulsion (Butyl acrylate/2- | 30 |
| ethylhexyl acrylate/methyl | |
| methacrylate = 40/30/30, | |
| solid content 50% by weight) | |
| Benzalkonium chloride | 0.02 |

### Example 3

Oil-in-Water Mascara

| Ingredient | Amount, weight percent (wt %) |
|---|---|
| Latanoprost | 0.005 |
| Vinyl acetate/vinyl pyrrolidone | 38.00 |
| copolymer¹ | |
| Hydroquinone | 0.5 |
| Deionized water | q.s. to 100% |
| Paraffin wax | 7.00 |
| Stearic acid | 9.00 |
| Triethanolamine | 1.50 |
| Iron oxide black | 8.50 |
| Ammonium acrylates copolymer | 10.00 |
| emulsion² | |

¹PVP/VA Copolymer W735 from International Specialty Products
²Syntran 5170, containing 41% by weight waterinsoluble polymer solids, available from Interpolymer Corp.

### Example 4

Oil-in-Water Mascara

| Ingredient | Amount, weight percent (wt %) |
|---|---|
| Copolymer of vinyl alcohol and | 38.19 |
| poly(oxyalkylene)acrylate.sup.1 | |
| Latanoprost | 0.005 |
| Hydroquinone | 0.5 |
| Deionized water | 11.65 |
| Hectorite.sup.2 | 3.10 |
| Carnauba wax | 1.60 |
| Paraffin wax | 5.90 |
| Beeswax | 3.00 |
| Stearic acid | 2.10 |
| Triethanolamine | 0.70 |
| Petroleum distillate | 4.00 |
| Tall oil glycerides.sup.3 | 2.00 |
| Pentaerythrityl- hydrogenated | 2.00 |
| rosinate.sup.4 | |
| Iron oxide black | 10.00 |
| Ammonium acrylates copolymer | 12.68 |
| emulsion.sup.5 | |
| Propylene glycol | 1.40 |
| Glycerine | 0.50 |
| Panthenol | 0.28 |
| Preservatives | q.s. to 100.00% |

¹Vinex, available from Air Products and Chemicals, incorporated via a 16.22 w/w % stock solution in deionized water.
²Bentone EW available from Rheox Inc.
³Zonester 85 available from Arizona Chemical Co.
⁴Foral 105 available from Hercules Inc.
⁵Syntran 5170, containing 41% by weight waterinsoluble polymer solids, available from Interpolymer Corp.

### Example 5

Water-in-Oil Mascara

| Ingredient | Amount, weight percent (wt %) |
|---|---|
| Cyclomethicone¹ | 6.0 |
| Latanoprost | 0.005 |
| Hydroquinone | 0.5 |
| Cyclomethicone/Dimethicone | 9.0 |
| Copolyol² | |
| Silicone Treated Iron Oxide³ | 12.0 |
| Ammonium acrylates copolymer | 34.0 |
| emulsion⁴ | |
| Copolymer of vinyl alcohol and | 23.0 |
| poly(oxyalkylene)acrylate⁵ | |
| Polyethylene⁶ | 16.0 |

¹Dow Corning 244 fluid available from Dow Corning
²Dow Corning 3225C, supplied as a solution of dimethicone copolyol in cyclomethicone available from Dow Corning
³STNP available from US Cosmetics, Inc.
⁴Syntran 5170 available from Interpolymer Corp.
⁵Vinex, available from Air Products and Chemicals, incorporated via a 20.0 w/w % stock solution in deionized water.
⁶ACumist B6 available from Allied Signal Inc.

### Example 6

Hair Lotion

| Ingredient | Amount, weight percent (wt %) |
|---|---|
| Latanoprost | 0.10 |
| Hydroquinone | 0.5 |
| Propylene glycol | 30.00 |
| Ethyl alcohol | 40.00 |
| Water | q.s. for 100.00 |

This lotion is applied to the scalp one or two times daily at the rate of 1 ml per application, the scalp being lightly massaged to bring about the penetration of the active principle. The hair is subsequently dried in the open air. This lotion makes it possible to reduce hair loss and to promote hair regrowth.

### Example 7

Hair Lotion

| Ingredient | Amount, weight percent (wt %) |
|---|---|
| Hydroquinone | 0.5 |
| Latanoprost | 0.10 |
| Propylene glycol | 30.00 |
| Ethyl alcohol | 40.00 |
| Water | q.s. for 100.00 |

### Example 8

Wax/Water Mascara

| Ingredient | Amount, weight percent (wt %) |
|---|---|
| Beeswax | 6.00 |
| Paraffin wax | 13.00 |
| Hydrogenated jojoba oil | 2.00 |
| Water-soluble film-forming polymer | 3.00 |
| Triethanolamine stearate | 8.00 |
| Black pigment | 5.00 |
| Preservative | q.s. |
| Hydroquinone | 0.5 |
| Latanoprost | 0.10 |
| Water | q.s. for 100.00% |

The mascara is applied to the eyelashes like a conventional mascara with a mascara brush.

While the invention has been described in detail and with reference to specific examples thereof, it will be apparent to one skilled in the art that various changes and modifications can be made therein without departing from the spirit and scope thereof.

What is claimed is:

1. A topical composition for stimulating hair growth in a mammal comprising:
a hair growth stimulating agent and/or hair density increasing agent;
a hair and/or skin lightening and/or neutralization agent; and
a cosmetically, personal care, or pharmaceutically acceptable excipient, carrier or vehicle,
wherein the composition has the effect of increasing length and/or thickness and/or density, and/or combination thereof, of the hair without darkening the hair and/or skin and/or iris of the mammal, and preventing hair loss.

2. A topical composition for stimulating hair growth and/or slowing or preventing the loss of hair and/or increasing the thickness of the hair in a mammal comprising:
a hair growth stimulating agent and/or hair density increasing agent selected from the group consisting of prostaglandins, prostaglandin analogs, 15-PDGH inhibitors, and combinations thereof;
a hair and/or skin lightening and/or neutralization agent; and
a cosmetically or pharmaceutically acceptable excipient, carrier or vehicle,
wherein the composition has the effect of increasing length and/or thickness and/or slowing or preventing loss, or any combination thereof, of the hair without darkening the hair and/or skin and/or iris of the mammal, and preventing hair loss.

3. The composition of claim 1, wherein the hair growth stimulating agent is selected from the group consisting of prostaglandins, prostaglandin analogs, and combinations thereof.

4. The composition of claim 1, wherein the a hair growth stimulating agent and/or hair density increasing agent is selected from the group consisting of prostaglandin A2, prostaglandin F2, prostaglandin E1, prostaglandin E2, Arbaprostil, Carboprost, Enprostil, Bimatoprost, Bemeprost, Latanaoprost, Limaprost, Minoxidil, Misoprostol, Ornoprostil, Prostacyclin, Prostaglandin E1, Prostaglandin E2, Prostaglandin F2α, Rioprostil, Rosaprostol, Sulprostone, Travaprost, Trimoprostil, Viprostol, 15-PGDH inhibitors, and combinations thereof.

5. The composition of claim 1, wherein the lightening agent is selected from the group consisting of hydroquinone, citric acid, perborate or persulfate salts, kojic acid, ascorbic acid, hydrogen peroxide, urea peroxide, melamine peroxide, sodium perborate, sodium percarbonate, sodium persulfate, and combinations thereof.

6. The composition of claim 1, wherein the hair is selected from the group consisting of head hair, scalp hair, eyelashes, eyebrows, mustaches, beards, chest, legs, arms, and combinations thereof.

7. The composition of claim 1 in a form selected from the group consisting of application stick, mascara, eyebrow coloring, eyeliner, eye shadow or other eye lid cosmetics, facial or body powder, sun tanning lotions and creams and sprays, sunscreens and blocks, nail polish, mousse, sprays, styling gels, nail conditioner, whether in the form of creams, lotions, gels, ointments, emulsions, colloids, solutions, suspensions, compacts, solids, pencils, brushes, spray-on formulations, brush-on formulations and the like, bath and shower gels, shampoos, conditioners, cream rinses, hair dyes and coloring products, leave-on conditioners, lip balms, skin conditioners, hair sprays, soaps, body scrubs, exfoliants, astringents, depilatories and permanent or temporary waving solutions, antidandruff formulations, antisweat and antiperspirant compositions, shaving, preshaving and after shaving products, moisturizers, deodorants, cold creams, cleansers, skin gels, rinses, whether in solid, powder, liquid, cream, gel, ointment, lotion, emulsions, colloids, solutions, suspensions, or other form, gels, patches, creams, sprays, ointments, lotions, emulsions, colloids, solutions, suspensions, powders and the like, and combinations thereof.

8. The topical composition of claim 1, further comprising at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV blocking agents, cosmetic and pharmaceutical active principles other than the compounds of formula (I), and combinations thereof.

9. The topical composition of claim 1, further comprising at least one other active principle selected from the group consisting of proteins, protein hydrolysates, amino acids, polyols, urea, allantoin, sugars and sugar derivatives, plant extracts, hydroxy acids, retinol or tocopherol derivatives, 5-(n-octanoyl)salicylic acid, esters of hydroxy acids, phospholipids, and combinations thereof.

10. The topical composition of claim 1, wherein said at least one additional ingredient is a cleaning agent, hair conditioning agent, skin conditioning agent, hair styling agent, antidandruff agent, hair growth promoter, perfume, sunscreen, sunblock, pigment, moisturizer, film former, hair color, make-up agent, detergent, pharmaceutical, thickening agent, emulsifier, humectant, emollient, antiseptic agent, deodorant active, dermatologically acceptable carrier, surfactant, abrasive, absorbent, fragrance, coloring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, addition peptide, buffering agent, bulking agent, chelating agent, cosmetic biocide, denaturant drug astringent, external analgesic, polymer, quat, substantivity increasing agent, opacifying agent, pH adjuster, propellant, reducing agent, sequestrant, skin bleaching agent, skin lightening agent, skin-conditioning agent, skin soothing agent, skin healing agent, aloe vera, pantothenic acid and derivative thereof, allantoin, bisabolol, dipotassium glycyrrhizinate, skin treating agent, thickener, or a vitamin or derivative thereof and mixtures or combinations thereof.

11. The topical composition of claim 1 further comprising a plurality of additional ingredients and wherein at least one of said additional ingredients is a dermatologically acceptable carrier.

12. A delivery device for the targeted application to the hair of a mammal a therapeutically effective amount of the composition of claim 1.

13. The delivery device of claim 1, wherein the device is for the application of the composition to eyelashes, eyebrows, and/or scalp.

14. A method for stimulating hair growth in a mammal comprising the application to mammalian skin an effective amount of the composition of claim 1.

15. A method for stimulating hair growth in a mammal comprising the application to mammalian skin of an effective amount of a composition comprising:
a hair growth stimulating agent and/or hair density increasing agent;
a hair and/or skin lightening and/or neutralization agent; and
a cosmetically, personal care, or pharmaceutically acceptable excipient, carrier or vehicle,
wherein the composition has the effect of increasing length and/or thickness and/or density, and/or combination thereof, of the hair without darkening the hair and/or skin and/or iris of the mammal, and preventing hair loss.

16. A method for increasing the thickness, and length of hair which comprises applying to the eyelids in the immediate vicinity of the normal lash line, or on the eyelashes, a composition according to claim 1.

17. A method for stimulating the growth of natural eyelashes in a human subject which comprises: applying to the base of the eyelash, or on the eyelashes, a therapeutically effective amount of the composition of claim 1.

18. A method for stimulating the growth of eyelashes in a patient which comprises: applying to the base of the eyelash, or on the eyelashes, a therapeutically effective amount of the composition of claim 1, wherein the patient is being treated for cancer.

19. A method for stimulating the growth of eyelashes in a patient which comprises: applying to the base of the eyelash, or on the eyelashes, a therapeutically effective amount of the composition of claim 1, wherein the patient suffers from alopecia.

20. A method for increasing the thickness, density and/or length of hair of an eyebrow or eyebrows which comprises applying to the eyebrow or eyebrows a composition according to claim 1.

21. A method for stimulating the growth of a natural eyebrow or eyebrows in a human subject which comprises: applying to the eyebrow or eyebrows a therapeutically effective amount of the composition of claim 1.

22. A method for stimulating the growth of an eyebrow or eyebrows in a patient which comprises: applying to the eyebrow or eyebrows a therapeutically effective amount of the composition of claim 1, wherein the patient is being treated for cancer.

23. A method for stimulating the growth of an eyebrow or eyebrows in a patient which comprises: applying to the eyebrow or eyebrows a therapeutically effective amount of the composition of claim 1, wherein the patient suffers from alopecia.
